(19) United States
(12) Patent Application Publication
(10) Pub. No.: US 2026/0072186 A1
(43) Pub. Date: Mar. 12, 2026

(54) DETECTOR STRUCTURES INCLUDING AN ANTI-SCATTER GRID WITH UNIFORM SUB-PIXEL SHIELDING AND METHODS OF FABRICATION THEREOF

(71) Applicant: Redlen Technologies, Inc., Saanichton (CA)

(72) Inventors: Michael K. JACKSON, Victoria (CA); Yuxin SONG, Victoria (CA); Krzysztof INIEWSKI, Port Moody (CA); Hiroaki NAKAI, Vancouver (CA)

(21) Appl. No.: 19/313,092

(22) Filed: Aug. 28, 2025

Related U.S. Application Data
(60) Provisional application No. 63/693,511, filed on Sep. 11, 2024.

(51) Int. Cl. G01T 1/24; A61B 6/03; A61B 6/42

(52) U.S. Cl. CPC G01T 1/244; A61B 6/032; A61B 6/4241

(57) ABSTRACT

Detector structures and methods of fabrication of detector structures that include a radiation sensor having a plurality of macropixels, each including a plurality of sub-pixels, and an anti-scatter grid (ASG) located over the radiation sensor, where the ASG has a pitch between adjacent septa of the ASG along at least one dimension that is less than 800μm, and the septa of the ASG partially shield an equal number of peripheral edges of each sub-pixel of each of the macropixels. In various embodiments, the ASG includes a plurality of first septa having a first vertical height dimension and a plurality of second septa having a second vertical height dimension different from the first vertical height dimension.

### FIELD

The present disclosure relates generally to radiation detectors, and more specifically to detector structures including a pixelated radiation sensor and an anti-scatter grid with uniform sub-pixel shielding and methods of fabrication thereof.

### BACKGROUND

Room temperature pixelated radiation detectors made of semiconductors, such as cadmium zinc telluride (Cd₁₋ₓZnₓTe where 0<x<1, or “CZT”), are gaining popularity for use in medical and non-medical imaging. These applications use the high energy resolution and sensitivity of the radiation detectors.

### SUMMARY

According to an aspect of the present disclosure, a detector structure includes a pixelated radiation sensor having a plurality of macropixels, each macropixel comprised of a plurality of sub-pixels, and an anti-scatter grid (ASG) located over the pixelated radiation sensor, where a pitch between adjacent septa of the ASG along a first horizontal direction is less than 800 μm, and the septa of the ASG shield an equal number of peripheral edges of each sub-pixel of each of the macropixels of the pixelated radiation sensor.

Additional embodiments include X-ray imaging systems including a radiation source configured to emit an X-ray beam, and a detector array including an above-described detector structure that is configured to receive the X-ray beam from the radiation source through an intervening space configured to contain an object therein.

Further embodiments include a detector structure, comprising a pixelated radiation sensor comprising a plurality of macropixels, each macropixel comprised of a plurality of sub-pixels, and an anti-scatter grid (ASG) located over the pixelated radiation sensor. The ASG comprises a plurality of first septa having a first vertical height dimension and a plurality of second septa having a second vertical height dimension; the first vertical height dimension is greater than the second vertical height dimension; and the second septa contact a surface of the radiation sensor or the second septa protrude into a lateral space between the first septa.

Further embodiments include a method of fabricating a detector structure that includes forming an X-ray attenuating material over a surface of a pixelated radiation sensor, and patterning the X-ray attenuating material located over the surface of the pixelated radiation sensor to form a plurality of septa contacting the pixelated radiation sensor, where each septum extends between and partially shields pixel detectors of the pixelated radiation sensor.

### BRIEF DESCRIPTION OF THE DRAWINGS

FIG. 1A is a functional block diagram of an X-ray imaging system in accordance with various embodiments of the present disclosure.

FIG. 1B is a schematically illustration of an application specific integrated circuit (ASIC) configured to count X-ray photons detected in each pixel detector within a set of energy bins according to various embodiments of the present disclosure.

FIG. 2A is a rear perspective view of a detector array for a computed tomography (CT) X-ray imaging system according to various embodiments of the present disclosure.

FIG. 2B is a perspective view of a CT X-ray imaging system illustrating the orientation of the detector array with respect to an X-ray source and a patient being imaged according to various embodiments of the present disclosure.

FIG. 3A is a top view illustrating a portion of a radiation sensor and a one-dimensional (1D) anti-scatter grid (ASG) over the front side of the radiation sensor.

FIG. 3B is a top view illustrating a portion of a radiation sensor and a two-dimensional (2D) anti-scatter grid (ASG) over the front side of the radiation sensor.

FIG. 3C is a side cross-sectional view of a radiation sensor and ASG taken along line A-A′ in FIGS. 3A and 3B.

FIG. 4A is a top view illustrating a portion of a radiation sensor including a macropixel having a 2×2 array of sub-pixels and a 1D ASG over the front side of the radiation sensor according to various embodiments of the present disclosure.

FIG. 4B is a top view of a detector structure including a radiation sensor having a macropixel with a 2×2 array of sub-pixels and a 2D ASG over the front side of the radiation sensor according to various embodiments of the present disclosure.

FIG. 4C is a top view of a detector structure including a radiation sensor having a macropixel with a 2×3 array of sub-pixels and a 1D ASG over the front side of the radiation sensor according to various embodiments of the present disclosure.

FIG. 4D is a side cross-sectional view of a detector structure including a radiation sensor and ASG taken along line B-B′ in FIGS. 4A, 4B or 4C according to various embodiments of the present disclosure.

FIG. 5 is a side cross-sectional view of a detector structure including a radiation sensor and an ASG having septa with different vertical height dimensions according to various embodiments of the present disclosure.

FIG. 6 is a side cross-sectional view of a detector structure including a radiation sensor and an ASG having first and second septa, where the second septa extend below the plane of the lower surfaces of the first septa, according to various embodiments of the present disclosure.

FIG. 7 is a side cross-sectional view of a detector structure including a radiation sensor and an ASG having first and second septa, where the second septa contact the upper surface of the radiation sensor according to various embodiments of the present disclosure.

FIG. 8 is a side cross-sectional view of a detector structure including a radiation sensor and an ASG including septa having a tapered shape according to various embodiments of the present disclosure.

FIG. 9 is a side cross-sectional view of a detector structure including a radiation sensor and an ASG including septa having non-planar sidewalls according to various embodiments of the present disclosure.

### DETAILED DESCRIPTION

Embodiments of the present disclosure provide detector structures for detecting ionizing radiation, the various aspects of which are described herein with reference to the drawings.

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims. Any reference to claim elements in the singular, for example, using the articles “a,” “an,” or “the” is not to be construed as limiting the element to the singular. The terms “example,” “exemplary,” or any term of the like are used herein to mean serving as an example, instance, or illustration. Any implementation described herein as an “example” is not necessarily to be construed as preferred or advantageous over another implementation. The drawings are not drawn to scale. Multiple instances of an element may be duplicated where a single instance of the element is illustrated, unless absence of duplication of elements is expressly described or clearly indicated otherwise.

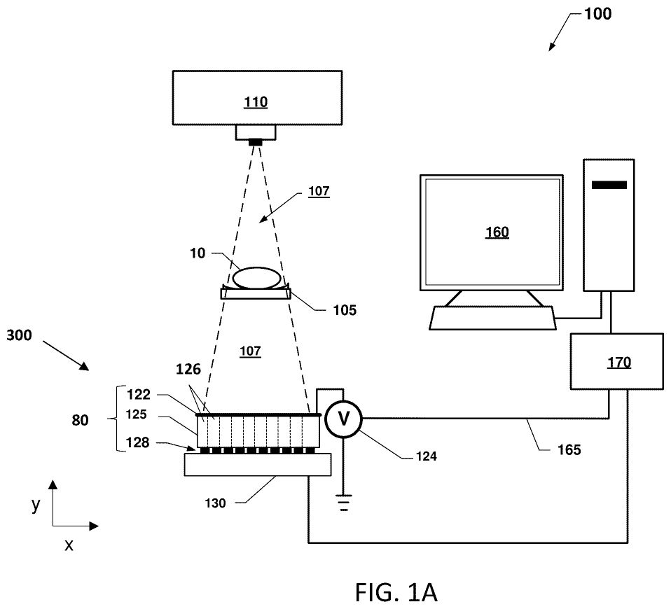

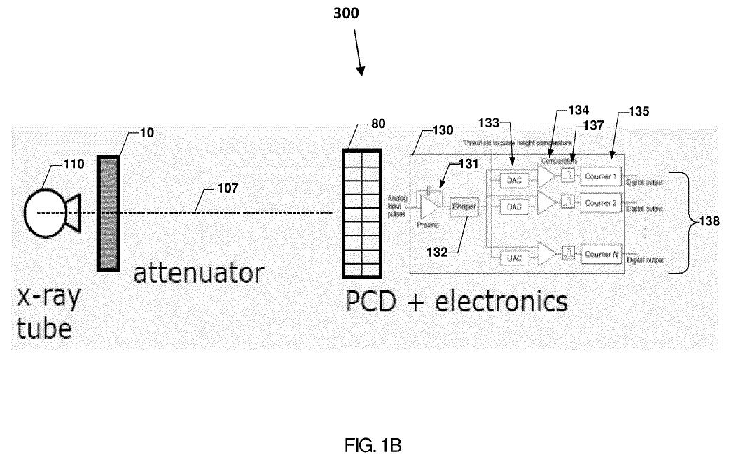

FIG. 1A is a functional block diagram of an X-ray imaging system 100 in accordance with various embodiments. The X-ray imaging system 100 may include an X-ray source 110 (i.e., a source of ionizing radiation), and an energy discriminating photon counting radiation detector array 300. The X-ray imaging system 100 may additionally include a patient support structure 105, such as a table or frame, which may rest on the floor and may support an object 10 to be scanned. In some embodiments, the object 10 may be a biologic subject (i.e., a human or animal patient). The support structure 105 may be stationary (i.e., non-moving) or may be configured to move relative to other elements of the X-ray imaging system 100, such as the X-ray source.

The X-ray source 110 is typically mounted to a gantry and may move or remain stationary relative to the object 10. The X-ray source 110 is configured to deliver ionizing radiation to the radiation detector array 300 by emitting an X-ray beam 107 toward the object 10 and the radiation detector array 300. After the X-ray beam 107 is attenuated by the object 10, the beam of radiation 107 is received by the radiation detector array 300.

The radiation detector array 300 may include one or more radiation sensors 80 coupled to detector read-out circuitry 130. Each radiation sensor 80 may be controlled by a high voltage bias power supply 124 that selectively creates an electric field between an anode 128 and cathode 122 pair coupled thereto. In one embodiment, each radiation sensor 80 includes a plurality of anodes 128 (e.g., one anode per pixel) and one common cathode 122 electrically connected to the power supply 124 and facing the X-ray source 110. Each radiation sensor 80 may include a detector material 125, such as a semiconductor material disposed between the anode 128 and cathode 122 and thus configured to be exposed to the electrical field therebetween. The semiconductor material may comprise any suitable semiconductor material for detecting X-ray radiation disposed between the anode 128 and cathode 122 and thus configured to be exposed to the electrical field therebetween. In various embodiments, the semiconductor material of the radiation sensor(s) 80 may comprise a II-VI semiconductor material, such as cadmium telluride, cadmium zinc telluride (i.e., CdZnTe or “CZT”), cadmium selenide telluride, and cadmium zinc selenide telluride. Other suitable “direct conversion” sensor materials, such as Si, Ge, GaAs, perovskites, etc., are within the contemplated scope of disclosure.

The detector read-out circuitry may include one or more application specific integrated circuits (ASICs) 130. Each ASIC 130 may be coupled to one or more radiation sensors 80 and may receive signals (e.g., charge or current) from the anodes 128 of the radiation sensor(s) 80. Each ASIC 130 may be configured to provide data to and by controlled by a control unit 170. Each of the radiation sensors 80 may be segmented or configured into a large number of small “pixel” detectors 126. In various embodiments, the pixel detectors 126 of the radiation sensors 80 and the ASIC(s) 130 are configured to output data that includes counts of photons detected in each pixel detector 126 in each of a number of energy bins. Thus, radiation detector arrays 300 of various embodiments may provide both two-dimensional detection information regarding where photons were detected, thereby providing image information, and measurements of the energy of the detected X-ray photons. A radiation detector array 300 that is capable of measuring the energy of the X-ray photons impinging on the array 300 may be referred to as an energy-discriminating radiation detector array 300.

The control unit 170 may be configured to synchronize the X-ray source 110, the read-out ASIC(s) 130, and the high voltage bias power supply 124. The control unit 170 may be coupled to and operated from a computing device 160. Alternatively, the computing device 160 and the control unit 170 may be integrated together as one device.

In some embodiments, the X-ray imaging system 100 may be a computed tomography (CT) imaging system. The CT imaging system 100 may include a gantry (not shown in FIG. 1A), which may include a moving part, such as a circular, rotating frame with the X-ray source 110 mounted on one side and the radiation detector array 300 mounted on the other side. The radiation detector array 300 may have a curved shape along its long axis (i.e., the x-axis direction in FIG. 1A) such that each of the pixel detectors 126 along the length of the radiation detector may face towards the focal spot of the X-ray source 110. The gantry may also include a stationary (i.e., non-moving) part, such as a support, legs, mounting frame, etc., which rests on the floor and supports the moving part. The X-ray source 110 may emit a fan-shaped or cone-shaped X-ray beam 107 as the X-ray source 110 and the radiation detector array 300 rotate on the moving part of the gantry around the object 10 to be scanned. After the X-ray beam 107 is attenuated by the object 10, the X-ray beam 107 is received by the radiation detector array 300. The curved shape of the radiation detector array 300 may allow the CT imaging system 100 to create a 360° continuous circular ring of the image of the object 10 by rotating the moving part of the gantry around the object 10.

For each complete rotation of the X-ray source 110 and the radiation detector array 300 around the object 10, one cross-sectional slice of the object 10 may be acquired. As the X-ray source 110 and the radiation detector array 300 continue to rotate, the radiation detector array 300 may take numerous snapshots called “views”. Typically, about 1,000 profiles are taken in one rotation of the X-ray source 110 and the radiation detector array 300. The X-ray source 110 and the detector array 300 may slowly move relative to the patient along a horizontal direction (i.e., into and out of the page in FIG. 1A) so that the detector array 300 may capture incremental cross-sectional profiles over a region of interest (ROI) of the object 10, which may include the entire object 10. The data acquired by the radiation sensor(s) 80 and output by the read-out ASIC(s) 130 may be passed along to the computing device 160 that may be located remotely from the radiation detector array 300 via a connection 165. The connection 165 may be any type of wired or wireless connection. If the connection 165 is a wired connection, the connection 165 may include a slip ring electrical connection between any structure (e.g., gantry) supporting the radiation detector array 300 and a stationary support part of the support structure, which supports any part (e.g., a rotating ring). If the connection 165 is a wireless connection, the radiation detector array 300 may contain any suitable wireless transceiver to communicate data with another wireless transceiver that is in communication with the computing device 160. The computing device 160 may include processing and imaging applications that analyze each profile obtained by the radiation detector array 300, and a full set of profiles may be compiled to form a three-dimensional computed tomographic (CT) reconstruction of the object 10 and/or two-dimensional images of cross-sectional slices of the object 10.

Various alternatives to the design of the X-ray imaging system 100 of FIG. 1A may be employed to practice embodiments of the present disclosure. X-ray imaging systems may be designed in various architectures and configurations. For example, an X-ray imaging system may have a helical architecture. In a helical X-ray imaging scanner, the X-ray source 110 and radiation detector array 300 are attached to a freely rotating gantry. During a scan, a table moves the object 10 smoothly through the scanner, or alternatively, the X-ray source 110 and detector array 300 may move along the length of the object 10, creating helical path traced out by the X-ray beam. Slip rings may be used to transfer power and/or data on and off the rotating gantry. In other embodiments, the X-ray imaging system may be a tomosynthesis X-ray imaging system. In a tomosynthesis X-ray scanner, the gantry may move in a limited rotation angle (e.g., between 15 degrees and 60 degrees) in order to detect a cross-sectional slice of the object 10. The tomosynthesis X-ray scanner may be able to acquire slices at different depths and with different thicknesses that may be reconstructed via image processing.

FIG. 1B illustrates components of an X-ray imaging system, including components within the ASIC 130 configured to count X-ray photons detected in each pixel detector 126 within a set of energy bins. As used herein, the terms “energy bin” and “bin” refer to a particular range of measured photon energies between a minimum energy threshold and a maximum energy threshold. For example, a first bin may refer to counts of photons determined to have an energy greater than a threshold energy (referred to as a trigger threshold, e.g., 20 keV) and less than 40 keV, while a second bin may refer to counts of photons determined to have an energy greater than 40 keV and less than 60 keV, and so forth.

X-rays 107 from an X-ray source (e.g., X-ray tube) 110 may be attenuated by a target (e.g., an object 10, such as a human or animal patient) before interacting with the radiation detector material within the pixelated detector array 300. An X-ray photon interacting (e.g., via the photoelectric effect) with a pixelated radiation detector material generates an electron cloud within the material that is swept by an electric field to the anode electrode 128. The charge gathered on the anode creates a signal that is integrated by an amplifier 131, such as a charge sensitive amplifier (CSA) or another amplifier, such as trans impedance amplifier (TIA). There may be an amplifier 131 for each pixel detector 126 (e.g., for each anode 128) within the pixelated X-ray detector array 300. The voltage of the amplifier output signal may be proportional to the energy of the X-ray photon. The output signal of the amplifier may be processed by an analog filter or shaper 132.

The filtered output may be connected to the inputs of a number of analog comparators 134, with each comparator connected to a digital-to-analog converter (DAC) 133 that inputs to the comparator a DAC output voltage that corresponds to the threshold level defining the limits of an energy bin. The detector circuitry 130 may be configured so that after the amplifier voltage has stabilized (after the dead time), that voltage may be between two voltage thresholds set by two DACs 133, which determines the output of the comparators 134. Outputs from the comparators 134 may be processed through decision gates 137, with a positive output from a comparator 134 corresponding to a particular energy bin (defined by the DAC output voltages) resulting in a count added to an associated counter 135 for the particular energy bin. Periodically, the counts in each energy bin counter 135 are output as signals 138 to the control unit 170.

Other suitable configurations for the read-out electronics of the ASIC 130 are within the contemplated scope of disclosure. For example, in some configurations, the analog voltage signals from the amplifier may be converted to digital signals using an analog-to-digital converter (ADC) prior to being sorted into the respective energy bins.

The detector array 300 of an X-ray imaging system may include an array of radiation detector elements, referred to herein as pixel detectors. The signals from the pixel detectors may be processed by a pixel detector circuit (e.g., an above-described ASIC 130), which may sort detected photons into energy bins based on the energy of each photon or the voltage generated by the received photon. When an X-ray photon is detected, its energy is determined and the X-ray photon count for its associated energy bin is incremented. For example, if the detected energy of an X-ray photon is 24 kilo-electron-volts (keV), the X-ray photon count for the energy bin of 20-40 keV may be incremented. The number of energy bins may be three or more, such as four to twelve. In an illustrative example, an X-ray photon counting detector may have four energy bins: a first bin for detecting photons having an energy between 20 keV and 40 keV, a second bin for detecting photons having an energy between 40 keV and 60 keV, a third bin for detecting photons having an energy between 60 keV and 90 keV, and a fourth bin for detecting photons having an energy above 90 keV (e.g., between 90 keV and 120 keV). The greater the total number of energy bins, the better the material discrimination. The total number of energy bins and the energy range of each bin may be selectable by a user, such as by adjusting the threshold levels defining the limits of the respective energy bins in the read-out ASIC 130 as shown in FIG. 1B.

In various embodiments, a detector array 300 for an X-ray imaging system 100 as described above may include a plurality of pixel detectors 126 extending over a continuous two-dimensional (2D) detector array surface. A typical radiation detector array 300 may include an array of individual radiation sensors 80 (e.g., a plate-like detector material 125 including cathode and anode electrode(s) 122, 128 defining pixel detectors 126 as described above) arranged side-by-side to provide the continuous 2D detector array surface. The detector array 300 (which is also known as a detector module system (DMS)) may further include a modular configuration including a plurality of detector modules, where each detector module may include at least one above-described radiation sensor 80, at least one ASIC 130 (also known as a read-out integrated circuit (ROIC)) electrically coupled to the at least one radiation sensor, and a module circuit board. The module circuit board may support transmission of electrical power, control signals, and data signals between the module circuit board and the at least one ASIC 130 and the at least one radiation sensor 80 of the detector module, and may further support transmission of electrical power, control signals, and data signals between the module circuit board and the control unit 170 of the X-ray imaging system 100, other module circuit boards of the detector array, and/or a power supply for the detector array. A plurality of detector modules may be assembled on a common support structure, such as a detector array frame, to form a detector array 300.

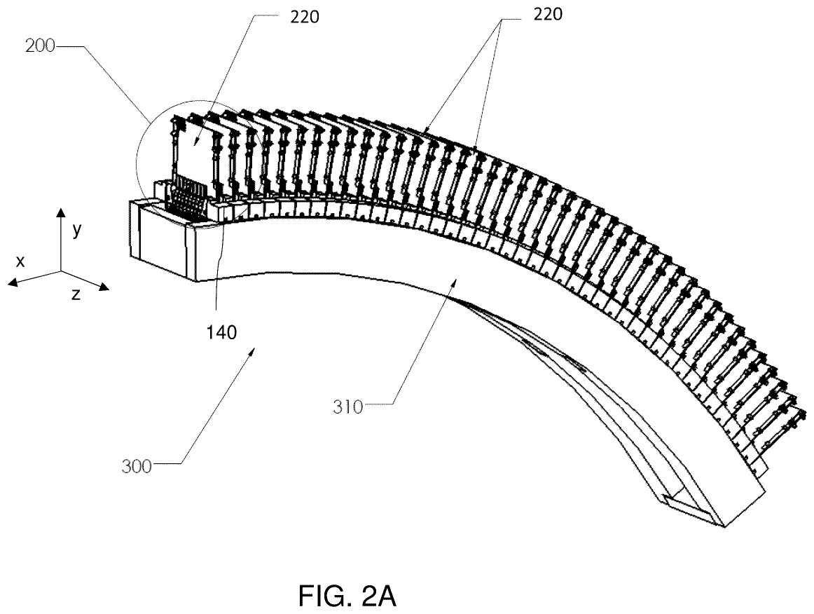

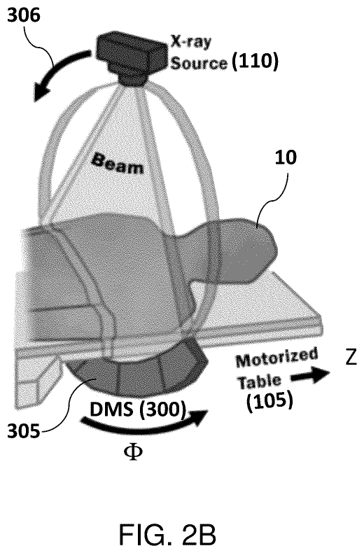

FIG. 2A is a rear perspective view of a detector array 300 for a computed tomography (CT) X-ray imaging system according to various embodiment of the present disclosure. The detector array 300 in this embodiment includes multiple detector modules 200 mounted on a detector array frame 310. The detector array frame 310 may be configured to provide attachment of a row of detector modules 200 such that physically exposed surfaces of the radiation sensors of the detector modules 200 collectively form a curved detection surface located within a cylindrical surface. The multiple detector modules 200 may be assembled such that radiation sensors attached to neighboring detector modules 200 abut each other, i.e., make direct surface contact with each other and/or include a gap between adjacent radiation sensors that is less than 3 mm, and/or less than 2 mm, and/or less than 1 mm in the x-direction. In some embodiments, the detector modules 200 may be mounted to the detector array frame 310 by attaching frame bars 90 of the detector modules 200 to the detector array frame 310 using suitable mechanical fasteners. The radiation sensors and ASICs 130 of each module 200 may be mounted over a first (i.e., front) surface of the frame bar 90. Each module 200 may also include a module circuit board 220 extending away from a rear surface of the frame bar 90. Major surfaces of the module circuit boards 220 of the detector modules 200 may face each other in the detector array 300.

FIG. 2B is a perspective view of a CT X-ray imaging system 100 illustrating the orientation of the detector array 300 with respect to an X-ray source 110 and a patient 10 being imaged according to various embodiments of the present disclosure. Referring to FIG. 2B, the X-ray source 110 and the detector array 300 (e.g., DMS) may rotate around the patient 10 and the support structure (e.g., motorized table 105) along the direction of arrow 306 to obtain cross-sectional image profiles (or “slices”) of the patient 10. The X-ray source 110 and the detector array 300 may also be translated relative to the patient 10 (e.g., by moving the support structure 105 and the patient 10 with respect to the X-ray source 110 and the detector array 300 and/or by moving the X-ray source 110 and the detector array 300 along the length of the patient 10) along a horizontal direction to obtain cross-sectional image “slices” of different portions of the patient 10. The direction of the horizontal movement of the X-ray source 110 and the detector array 300 relative to the patient 10 may be referred to as the “Z-axis” direction, which may be parallel to the axis of rotation of the X-ray source 110 and the detector array 300 around the patient 10. As discussed above, the detector array 300 may also have a curved shape along the direction in which the X-ray source 110 and the detector array 300 rotate around the patient. The pixel detectors of the detector array 300 may be arranged in multiple columns and rows of pixel detectors, where each column may extend along the Z-axis direction, and each row may extend along the direction of rotation of the detector array 300 around the patient. Accordingly, the location of each pixel detector within the detector array 300 may be defined by a unique row and column pair, where the location of the pixel detector within a given column may be defined by its location along the Z-axis direction, and the location of the pixel detector within a given row may be defined by the azimuth angle Φ of a line segment extending between the pixel detector and the focal spot of the X-ray source 110, where all pixel detectors within the same column may have the same azimuth angle Φ. The detector array 300 shown in FIG. 2B may be similar to the detector array 300 described above with reference to FIG. 2A. The detector array 300 may further include a suitable housing or enclosure 305 that encloses and protects the module circuit boards 220.

Related X-ray imaging systems, including CT X-ray imaging systems, often utilize energy integrating (EI) detectors (EID). In EI detectors, the radiation-sensitive sensor material is commonly a solid-state scintillator material that is coupled to a photodiode. The scintillation light generated by the sensor material is proportional to both the energy of each photon incident on the sensor material as well as the number of incident photons per unit time. The photodiode converts the scintillation light to an electric signal that is amplified and integrated by the read-out electronics to produce the output signal.

In contrast, an imaging system 100 according to various embodiments may utilize a photon counting (PC) detector in which the detector utilizes a “direct conversion” sensor material that is configured to directly detect photon interactions occurring within the sensor material. The sensor material is coupled to read-out circuitry (e.g., an ASIC) that measures the total photon counts for different energy bins, as described above. In the case of a CT X-ray imaging system, the PC detector may also be referred to as a Photon Counting Computed Tomography (PCCT) detector.

X-ray imaging systems, such as X-ray CT imaging systems, utilizing EI detectors have been used for many years for medical and industrial imaging and other applications. EI detectors typically include detector pixels having a center-to-center spacing (i.e., pitch) of ˜1 mm (e.g., 1 mm±20%). The detectors may also utilize an anti-scatter grid (ASG) composed of a suitable x-ray absorbing material located over the surface of the sensor that is configured to reduce the number of scattered photons that reach the detector surface. The ASG may include a network of vertically extending partitions (i.e., septa) aligned over the front surface of the sensor and including openings between the partitions. The separation distance between adjacent septa of the ASG may correspond to the pixel pitch of the detector (e.g., ˜1 mm).

PC detectors, such as PCCT detectors, were developed more recently than EI detectors and inherited many of the characteristics of existing EI detectors, such as a ˜1 mm pitch between pixels and the corresponding size of the ASG. However, in a PC detector, the pixel size is typically a function of the size of the anode electrodes and the gaps (or “streets”) between the adjacent anode electrodes. Thus, a PC detector may divide each 1 mm pixel area into a number of smaller pixel detectors 126, which may be referred to as sub-pixels. Dividing a larger (e.g., ˜1 mm²) pixel area into multiple sub-pixels may provide enhanced spatial resolution, for example. An ASG having a conventional (e.g., ˜1 mm) separation distance between adjacent septa may be provided over the PC detector, where groups of adjacent sub-pixels that are exposed through the openings of the ASG may be referred to as “macropixels. ”

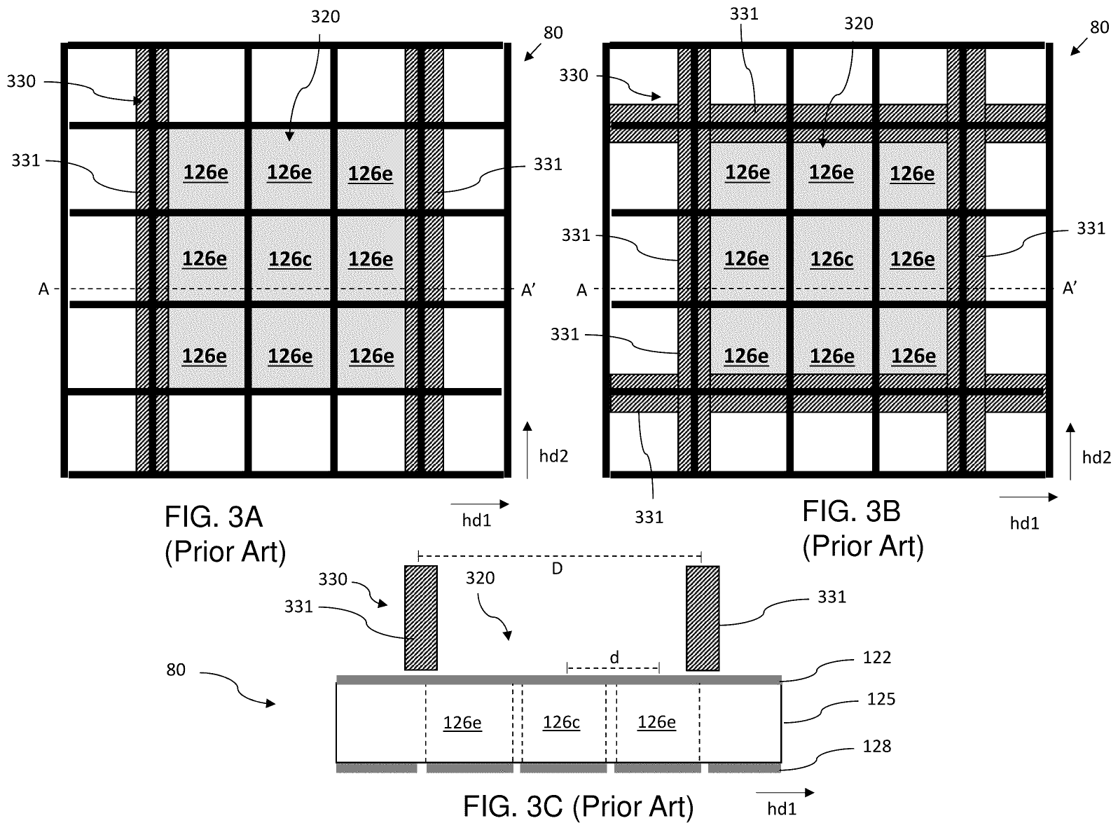

FIGS. 3A-3C illustrate prior art photon counting radiation detectors including a pixelated radiation sensor 80 and an anti-scatter grid (ASG) 330 over the front (i.e., X-ray source facing) side of the radiation sensor 80. FIG. 3A is a top view illustrating a portion of the radiation sensor 80 and a one-dimensional (1D) ASG 330 over the front side of the radiation sensor 80. FIG. 3B is a top view illustrating a portion of the radiation sensor 80 and a two-dimensional (2D) ASG 330 over the front side of the radiation sensor 80. FIG. 3C is a side cross-sectional view of a radiation sensor 80 and ASG 300 taken along line A-A′ in FIG. 3A or 3B. The 1D ASG 330 shown in FIG. 3A includes series of septa 331 that are spaced from one another along a first horizontal direction hd1 and that extend parallel to one another along a second horizontal direction hd2. The 2D ASG 330 shown in FIG. 3B includes septa 331 extending in a grid-like manner along two orthogonal horizontal directions, hd1 and hd2. FIGS. 3A and 3B illustrate the septa 331 as being partially-transparent to illustrate the position of the ASG 330 with respect to the underlying array of pixel detectors 126 of the radiation sensor 80. The ASG 330 may be mounted over the front side of the radiation sensor 80 such that there is a small gap between the ASG 330 and the cathode electrode 122 of the radiation sensor 80, as shown in FIG. 3C. The ASG 330 may be aligned over the radiation sensor 80 such that each septum 331 overlies gaps between adjacent anode electrodes 128 of the radiation sensor 80. The septa 331 of the ASG 330 may extend along edges of “macropixels” 320 composed of groups of individual pixel detectors 126 (i.e., “sub-pixels”) of the radiation sensor 80. FIGS. 3A and 3B illustrate a single macropixel 320 via darker shading. In the 1D ASG 330 shown in FIG. 3A, the septa 311 of the ASG 330 extend along two opposite edges of each macropixel 320. In the 2D ASG 300, the septa 311 of the ASG 330 may extend around all four edges of each of the macropixels 320.

The center-to-center distance between adjacent septa 331 of the ASG 330 may be 1 mm±20%, which may correspond to the typical ˜1 mm pixel pitch in prior IE detectors. FIG. 3C illustrates the center-to-center distance, D, between adjacent septa 331 of the ASG 330 along the first horizontal direction hd1. This distance, D, may be referred to as the “pitch” of the ASG 330. The 1D ASG 330 shown in FIG. 3A includes a pitch, D, of ˜1 mm along the first horizontal direction hd1. The 2D ASG shown in FIG. 3B may have an identical pitch, D, of ˜1 mm along both the first horizontal direction hd1 and the second horizontal direction hd2.

The size of each macropixel 320 may be equivalent to the pitch, D, of the ASG 330, which typically corresponds to the ˜1 mm pixel pitch in prior IE detectors. Thus, in the example shown in FIGS. 3A-3C, the size of each macropixel 320 may be ˜1 mm×1 mm, or ˜1 mm². When designing the layout of the pixel detectors 126 that form the “sub-pixels” of the macropixels 320, there are a limited number of practical ways to divide up the area of the macropixels 320. For example, each macropixel 320 may include a 2×2 set of pixel detectors/sub-pixels 126. For a ˜1 mm macropixel 320, this would provide pixel detectors 126 having a center-to-center spacing, or pitch, of ˜500 μm. Alternatively, a 3×3 set of pixel detectors 126 would provide a pitch of ˜330 μm, while a 4×4 set of pixel detectors 126 would provide a pitch of ˜250 μm. However, in high photon flux applications, such as PCCT imaging applications, a relatively large sub-pixel pitch of ˜500 μm may lead to excessive “pile-up” effects in which multiple photons may impinge upon the same pixel detector 126 during a single read-out cycle, which can result in distortions in detecting the number of photons and the energy of each photon. Conversely, a relatively small sub-pixel pitch of ˜250 μm may lead to excessive “charge sharing” effects, where photons strike near the boundaries of two or more sub-pixels, such that multiple sub-pixels may register a photon count for the same photon and the detected energy may not accurately reflect the actual energy of the photon. Consequently, a 3×3 arrangement of sub-pixels with a center-to-center spacing (i.e., D in FIG. 3C), or “pitch,” of ˜330 μm has become an industry norm for PCCT detectors due to this design being less affected by pile-up and charge sharing effects.

While a 3×3 arrangement of sub-pixels with a ˜330 μm pitch may help reduce the impact of pile-up and charge sharing effects as described above, this design may contribute to sub-optimal count stability in a PC detector. An ideal PC detector should have photon count values that depend solely upon the instantaneous spectrum and intensity of the radiation incident upon the detector. In practice, however, the detector response to a given radiation stimulus may vary over time. The stability of a radiation detector may be characterized by applying a step flux response and quantifying the count stability over a particular timeframe using a suitable metric. For example, a “1 minute stability” metric may measure the count rate changes at different intervals over a one-minute timeframe.

A 3×3 sub-pixel configuration as shown in FIGS. 3A-3C may exhibit sub-optimal count stability characteristics, including sub-optimal 1 minute stability characteristics, due to non-uniform “shadowing” of the sub-pixels by the ASG 330. Considering a macropixel 320 composed of a 3×3 rectangular array of sub-pixels as shown in FIGS. 3A-3C, the macropixel 320 includes one center sub-pixel (126c in FIGS. 3A-3C) that is surrounded on all sides by other sub-pixels of the macropixel 320, such as eight edge sub-pixels (126e in FIGS. 3A-3C) that are each adjacent to at least one peripheral edge of the macropixel 320. When a 1D ASG 330 is utilized as shown in FIG. 3A, six of the eight edge sub-pixels 326e (i.e., sub-pixels 326e along the left-and right-hand sides of the macropixel 320) are partially shielded (i.e., “shadowed”) from receiving incident radiation by the septa 331 of the ASG 330 while the center sub-pixel 126c and the pair of edge sub-pixels 126e located above and below the center sub-pixel 126c are unshielded by the ASC 330. In the case of a 2D ASG as shown in FIG. 3B, all eight of the edge sub-pixels 126e are partially shielded by the ASG 320 while the center sub-pixel 126c is unshielded.

This non-uniform shielding of the sub-pixels 126 of the macropixels 320 may result in a “lensing” effect where exposure to incident X-ray radiation may, over time, cause the electric fields within the sensor material 125 to shift in a non-uniform manner. In particular, in regions of the sensor material 125 that are exposed to incident radiation, the electric field tends to drop near the anode and increase near the cathode. However, in regions of the sensor material 125 that are shadowed by the septa 131 of the ASG 130, incident radiation is blocked and there is no change in electric field. Accordingly, the potential lines within the sensor material 125 may shift, with the net effect of the shifted potential lines “pushing” photoelectrons away from regions of the sensor material 125 that are shadowed by the septa 331. Accordingly, photon count values may increase for the un-shielded sub-pixels 126 (e.g., 126c) in the central regions of each macropixel 320 and may decrease for the partially shielded sub-pixels 126 (e.g., 126e) along the peripheral edges of each macropixel 320. This may negatively affect the count stability characteristics of the detector.

Various embodiments are directed to photon counting radiation detector structures including a radiation sensor 80 including a plurality of macropixels 320, each including a plurality of sub-pixels 126, and an anti-scatter grid (ASG) 330 over the radiation sensor 80, where the ASG 300 has a pitch D between adjacent septa 331 of the ASG 300 along at least one dimension that is less than 800 μm, such as ≤750 μm, including about 660 μm, and the septa 331 of the ASG 330 partially shields an equal number of peripheral edges of each sub-pixel 126 of each of the macropixels 320 of the radiation sensor. In various embodiments, each sub-pixel 126 may have a pitch d that is less than 400 μm, such as between 300 μm and 400 μm, including 330 μm±20%. In various embodiments, by providing macropixels 320 in which each sub-pixel 126 is partially shielded along the same number of peripheral edges by the ASG 330, the above-described “lensing” effect may be reduced or avoided, thereby providing improved count stability characteristics of the radiation detector. In addition, the sub-pixels 126 of each macropixel 320 may have a pitch (e.g., 330 μm±20%) that may minimize pileup and charge sharing effects.

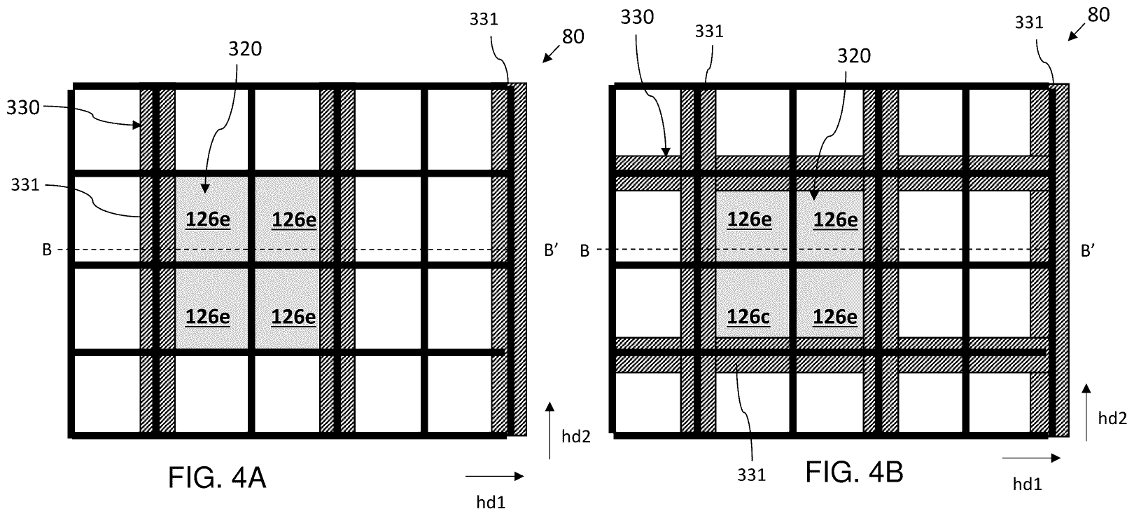

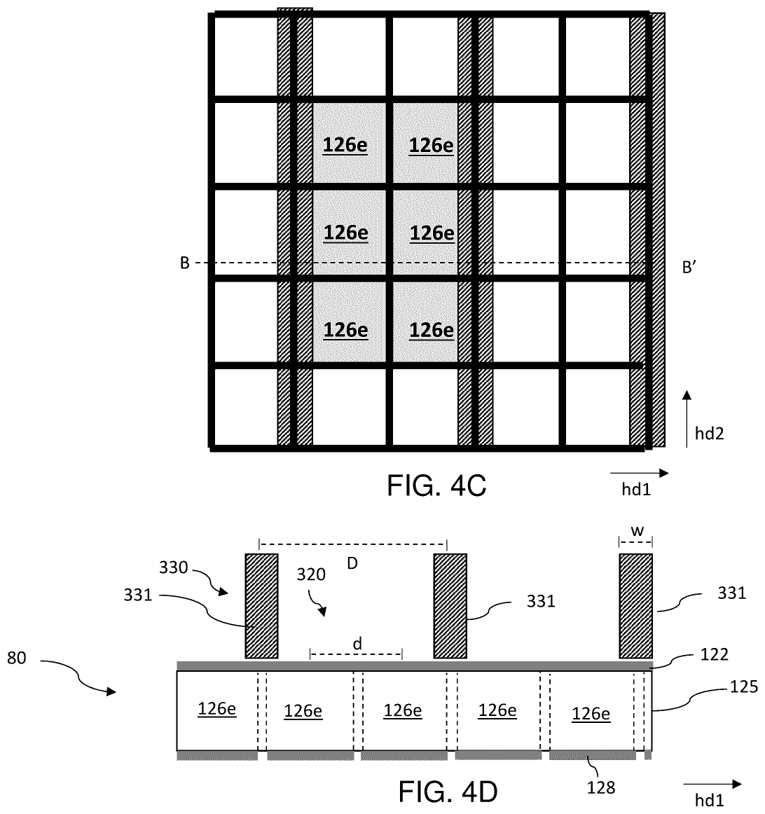

FIGS. 4A-4D illustrate photon counting radiation detectors according to various embodiments. FIG. 4A is a top view of a detector structure including a radiation sensor 80 including a macropixel 320 with a 2×2 array of sub-pixels 126 and a one-dimensional (1D) ASG 330 over the front side of the radiation sensor 80. FIG. 4B is a top view of a detector structure including a radiation sensor 80 having a macropixel 320 with a 2×2 array of sub-pixels 126 and a two-dimensional (2D) ASG 330 over the front side of the radiation sensor 80. FIG. 4C is a top view of a detector structure including a radiation sensor 80 having a macropixel 320 with a 2×3 array of sub-pixels 126 and a one-dimensional (1D) ASG 330 over the front side of the radiation sensor 80. FIG. 4D is a side cross-sectional view of a detector structure including a radiation sensor 80 and ASG 300 taken along line B-B′ in FIGS. 4A, 4B or 4C. In one non-limiting embodiment, the pitch between adjacent sub-pixels 126 in each macropixel 320 may be 330 μm±20%. The pitch of the ASG 330 (i.e., the center-to-center distance, D, between adjacent septa 331) may be 660 μm±20% along the first horizontal direction hd1 for both the 1D ASGs 330 shown in FIGS. 4A and 4C and the 2D ASG 330 shown in FIG. 4B. In some embodiments, the pitch of the ASG 330 may also be 660 μm±20% along the second horizontal direction hd2 for the 2D ASG 330 shown in FIG. 4B.

In various embodiments, the ASG 330 may partially shield (i.e., shadow) all of the sub-pixels 226 in each macropixel 320. In various embodiments, each macropixel 320 may have a width of two sub-pixels 126 along at least one horizontal direction (i.e., hd1 in FIGS. 4A-4D). In the 1D ASG 330 embodiments shown in FIGS. 4A and 4C, each sub-pixel 126 may be partially shielded by a septum 331 of the ASG 330 along one peripheral edge of the sub-pixel. In some embodiments including a 1D ASG 330, each macropixel 320 may include a width of greater than two sub-pixels along the direction parallel to the septa 331 of the ASG 330 (i.e., hd2), such as a 2×3 configuration of sub-pixels 126 as shown in FIG. 4C. In embodiments including a 2D ASG 330 such as shown in FIG. 4B, the macropixels 320 may include a 2×2 configuration of sub-pixels 126. Each sub-pixel 126 may be partially shielded by septa 331 of the ASG 330 along two peripheral edges of the sub-pixel 126. Accordingly, in various embodiments, each individual pixel detector 126 may form an edge sub-pixel 126e of a macropixel 300, and none of the pixel detectors 126 may be center sub-pixels 126c. As discussed above, by providing uniform shading of the sub-pixels such that an equal number of peripheral edges of each sub-pixel 126 is shielded by the ASG 330, the lensing effect that pushes photoelectrons from edge sub-pixels to the center sub-pixels may be mitigated and count stability performance of the detector may be improved. In addition, providing a relatively smaller ASG pitch (e.g., 660 μm vs. 1 mm in the comparative example of FIGS. 3A-3C), the ASG 330 may also provide improved scatter reduction.

One potential drawback of a reduced ASG pitch may be a loss of detector quantum efficiency (DQE) due to an increase of the “dead area” of the detector that is shielded by the ASG 330. In the comparative example of FIG. 3A including a 1D ASG 330 having a 1 mm ASG pitch and 330 μm sub-pixel pitch, the DQE loss resulting from sub-pixel shielding by the ASG 330 is 10%. However, in an embodiment 1D ASG 300 having the same 330 μm sub-pixel pitch but with a 660 μm ASG pitch, the DQE loss due to the ASG is 15%. This potential reduction in DQE may be at least partially offset by the design of the ASG 300. FIG. 4D illustrates an ASG 330 in which each of the septa 331 has a width dimension, W. Current ASGs 330 typically include septa 331 having a width of ˜100 μm. In various embodiments, the width dimension w of the septa 331 may be made smaller, such as ≤80 μm, for example 50 to 75 μm including about 67 μm. This may reduce the overall “dead area”of the detector and improve the DQE.

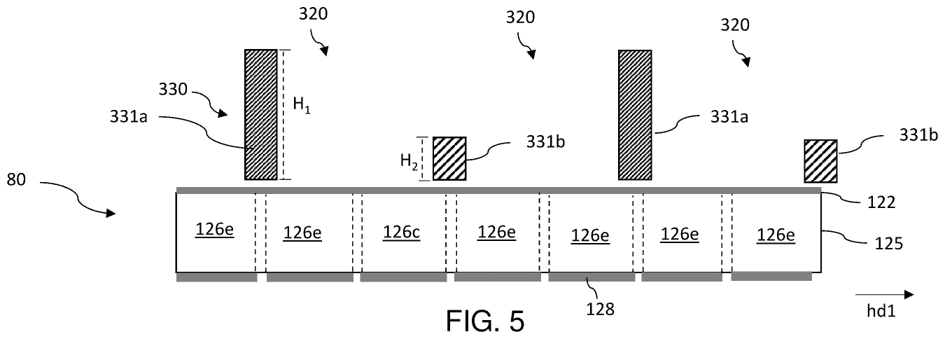

In the embodiment shown in FIGS. 4A-4D, each of the septa 331 of the ASG 330 may have the same size and shape and may be composed of the same material(s). In other embodiments, the septa 331 of the ASG 300 may have non-uniform characteristics. FIG. 5 is a side cross-section view of a detector structure including a radiation sensor 80 and an ASG 300 having septa with different vertical height dimensions. In various embodiments, the ASG 300 may include first septa 331a having a first vertical height dimension H₁ and second septa 331b having a second vertical height dimension H₂, where H₁ >H₂. In some embodiments, the first vertical height dimension H₁ may be greater than 2 mm, such as between about 2.5 mm and about 3 mm. The second vertical height dimension H₂ may be 1 mm or less, such as between about 0.1 mm and 1 mm (e.g., ˜0.5 mm). The first septa 331a and the second septa 331b may alternate with one another along the first horizontal direction hd1. In embodiments including a 2D ASG 300 such as shown in FIG. 4B, the ASG 300 may similarly include septa having different vertical height dimensions that may alternate with one another along the second horizontal direction hd2. Each macropixel 320 may include at least one first septa 331a extending along a peripheral edge of the macropixel 320 and at least second septa 331b extending along an opposite edge of the macropixel 320. In various embodiments, providing an ASG 330 including relatively taller septa 331a alternating with relatively shorter septa 331b may reduce the costs of the ASG.

In some embodiments, the first septa 331a and the second septa 331b may be physically connected to one another, such as around the periphery of the ASG 330, by transverse septa in the case of a 2D ASG 330, and/or by a connecting matrix between the septa 331a, 332b composed of low X-ray attenuating material. Alternatively, the first septa 331a and the second septa 331b may not be physically connected to each other. The first septa 331a and the second septa 331b may have the same width dimensions, W, or may have different width dimensions.

The first septa 331a and the second septa 331b may be composed of the same material, or may be composed of different materials. In some embodiments, the choice of materials and the vertical heights of the septa 331a, 331b may be selected to help balance the photon absorption characteristics of the respective septa 331a. For example, the first septa 331a may include lead or a lead alloy material and may have a vertical height, H₁, of 2-3 mm, as is typical of ASGs currently used in X-ray imaging systems. The second septa 331b may have a combination of vertical height and material characteristics that may be capable of stopping a predetermined percentage (e.g., 90%) of X-ray photons from passing through the septa 331b and reaching the surface of the detector. For example, a second septum 331b composed of tungsten (W) may have a minimum height dimension H₂ of 0.1 to 0.2 mm, such as about 0.12 mm, to stop at least 90% of X-rays having an average photon energy of 74 keV. In the case of lead (Pb), the second septum 331b would need a minimum height dimension H₂ of ˜0.71 mm to stop at least 90% of X-rays having an average photon energy of 74 keV. In the case of molybdenum (Mo), the second septum 331b would need a minimum height dimension H₂ of ˜0.71 mm to stop at least 90% of X-rays having an average photon energy of 74 keV.

In the embodiment shown in FIG. 5, the lower surfaces of the second septa 331b are substantially co-planar with the lower surfaces of the first septa 331a. The second septa 331b are located in spaces between the first septa 331a. In other words, a horizontal plane parallel to the top surface of the cathode electrode 122 which extends from a sidewall of one first septa 331a to a sidewall of another first septa 331a cuts through a second septa 331b located in the lateral space between the two first septa 331.

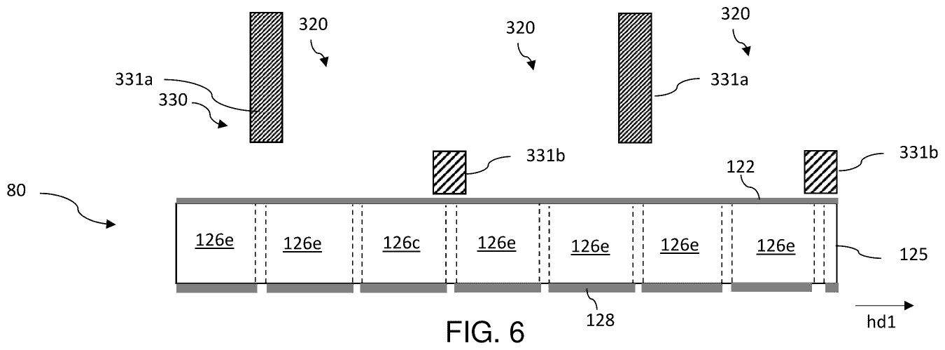

FIG. 6 is a side cross-section view of a detector structure including a radiation sensor 80 and an ASG 300 having first and second septa 331a, 331b, where the second septa 331b extend below the plane of the lower surfaces of the first septa 331a. In some embodiments, the second septa 331b may be entirely below the plane of the lower surfaces of the first septa 331a. This may, for example, facilitate the provision of a 2D ASG 300 where the second septa 331b may form a two-dimensional grid structure that underlies a two-dimensional grid structure of first septa 331a, and the second septa 331b are offset from the first septa 331a by the width of one sub-pixel of the radiation sensor 80.

In still further embodiments, the ASG 300 may include a two-dimensional grid in which the first septa 331a may all extend parallel to one another along a first direction (e.g., hd1) and the second septa 331b may all extend parallel to one another along a second direction (e.g., hd2). Each macropixel 320 may be surrounded on two opposing sides by a first septa 331a and on the other two opposing sides by a second septa 331b.

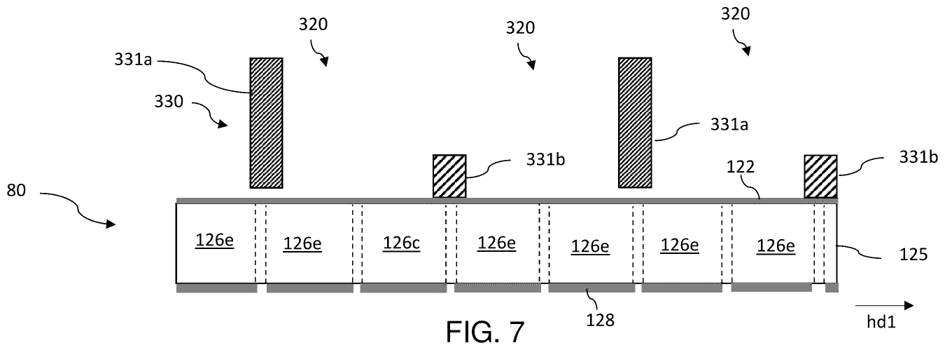

In the embodiments shown in FIGS. 4A-6, the ASG 330 is vertically offset from the radiation sensor 80 such that none of the septa contact the cathode-side of the radiation sensor 80. In other embodiments, all or a portion of the septa of the ASG 330 may contact the cathode-side of the radiation sensor 80. FIG. 7 is a side cross-section view of a detector structure including a radiation sensor 80 and an ASG 300 having first and second septa 331a, 331b where the second septa 331b contact the upper surface (i.e., the cathode-side surface, such as the cathode electrode 122) of the radiation sensor 80. In various embodiments, the second septa 331b may not physically contact the first septa 331a of the ASG 300 to avoid electrically shorting the cathode electrode 122 to the first septa 331a.

In some embodiments, the second septa 331b may be pre-fabricated and placed onto the upper surface of the radiation sensor 80. In other embodiments, the second septa 331b may be fabricated in situ on the upper surface of the radiation sensor 80. In one embodiment, a material having suitable X-ray attenuation characteristics may be formed as a continuous layer over the upper surface of the radiation sensor 80 and may be patterned to form discrete second septa 331b of the ASG 330. Suitable X-ray attenuating materials may include, for example, a metal material, such as tungsten (W), lead (Pb), and/or molybdenum (Mo). Other suitable X-ray attenuating materials are within the contemplated scope of disclosure. The continuous layer of material may be deposited using a suitable deposition method, such as via physical vapor deposition (PVD) (e.g., sputtering), chemical vapor deposition (CVD), atomic layer deposition (ALD), or the like. The layer may be patterned by forming a photoresist layer (not shown in FIG. 7) over the layer of X-ray attenuating material and lithographically patterning the photoresist layer to form a mask in a desired pattern. An etching process may be used to remove portions of the continuous layer of X-ray attenuating material that are exposed through the mask to form the discrete second septa 331b over the upper surface of the radiation sensor 80. Other suitable method, such as a lift-off method, may be used to form the second septa 331b. In a lift-off method, the photoresist layer is formed on the cathode electrode 122 and then patterned. The X-ray attenuating material is then deposited over the photoresist pattern and on the cathode electrode 122 in spaces between the photoresist pattern. The photoresist pattern is then lifted off the cathode electrode along with portions of the X-ray attenuating material deposited on top of the photoresist pattern, to leave a pattern of the second septa 331b on the cathode electrode 122.

First septa 331a may be provided over the upper surface the radiation sensor 80 to provide an ASG 330 as described above. The first septa 331a may be vertically offset from the upper surface of the radiation sensor 80 (e.g., from the cathode electrode 122) and may be laterally offset from second septa 331b.

ASGs 300 typically include fabrication errors, such as with respect to their flatness (e.g., if they are made from patterned sheets of metal) and/or their surface roughness (e.g., if 3D printed). As a consequence of these “shape errors,” the effective shadowing on the detector may not be equal to the width of the septa at any given point (the “nominal width”), but rather may be equal to an “effective width”based on the nominal width and the shape errors.

Various embodiments may include ASGs 330 in which all or a portion of the septa 331 have non-vertical sidewalls. In various embodiments, this may enable the septa 331 to shadow a larger effective width of the radiation sensor 80 than the nominal width of the septa 331. Accordingly, the ASG 300 may be fabricated with less material, which may reduce costs.

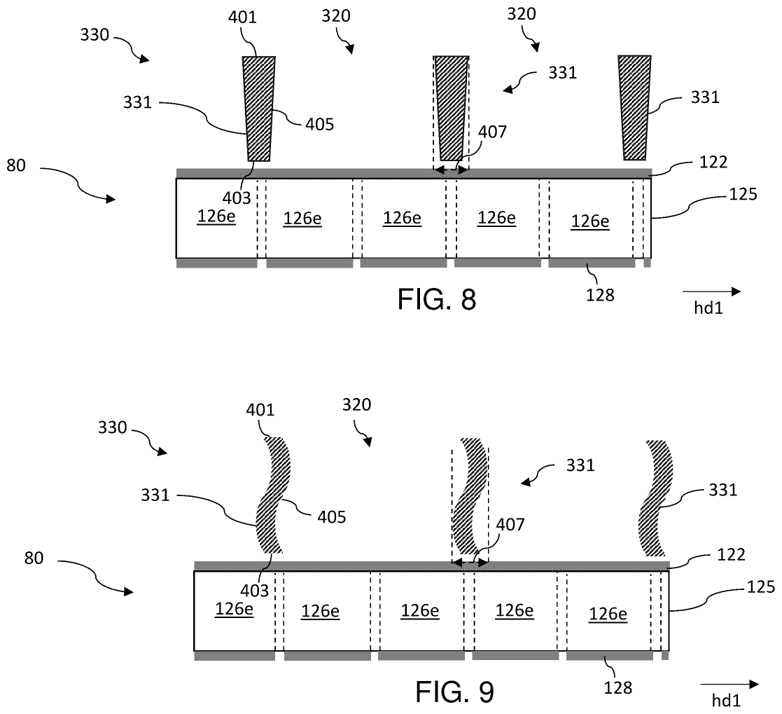

FIG. 8 is a side cross-section view of a detector structure including a radiation sensor 80 and an ASG 300 including septa 331 having a tapered shape. The detector structure may be similar to the detector structure described above with reference to FIGS. 4A-4D. Thus, repeated discussion of like features is omitted for brevity. The ASG 330 in the embodiment of FIG. 8 includes septa 331 having a larger width dimension on the upper surfaces 401 of the septa 331 than on the lower surfaces 403 of the septa 331. Sidewalls 405 of the septa 331 may be angled or curved with respect to vertical such that the width of each septum 331 tapers between the upper surface 401 and the lower surface 403. The relatively wider upper portions of each septum 331 may shield the underlying surface of the radiation sensor 80 from incident radiation over a larger effective width 407 than the nominal width of the septum 331 on the lower surface 403 of the septum 331. Although FIG. 8 illustrates the septa 331 having uniform sizes and shapes, it will be understood that septa 331 having tapered shapes as shown in FIG. 8 may be utilized for any of the septa 331a, 331b in the embodiments shown in FIGS. 5-7.

FIG. 9 is a side cross-section view of a detector structure including a radiation sensor 80 and an ASG 300 including septa 331 having non-planar sidewalls 405. In the embodiment shown in FIG. 9, the septa 331 may have a substantially uniform width dimension between the upper surfaces 401 and the lower surfaces 403. However, the sidewalls 405 may be non-planar sidewalls 405 that may be curved or angled with respect to a vertical plane. In the embodiment shown in FIG. 9, the sidewalls 405 are curved sidewalls that form an “S” shape of the septum 331 when viewed in a side cross-section. The non-planar sidewalls 405 of each septum 331 may shield the underlying surface of the radiation sensor 80 from incident radiation over a larger effective width 407 than the nominal width of the septum 331. It will be understood that septa 331 having non-planar sidewalls 405 as shown in FIG. 9 may be utilized for any of the septa 331a, 331b in the embodiments shown in FIGS. 5-7.

The devices of the embodiments of the present disclosure can be employed in various radiation detection systems including computed tomography (CT) imaging systems. Any direct conversion radiation sensors may be employed such as radiation sensors employing Si, Ge, GaAs, CdTe, CdZnTe, and/or other similar semiconductor materials.

The detector structures of the present embodiments may be used for medical imaging, such as in Low-Flux applications in Nuclear Medicine (NM), whether by Single Photon Emission Computed Tomography (SPECT) or by Positron Emission Tomography (PET), or as radiation detectors in High-Flux applications as in X-ray Computed Tomography (CT) for medical applications, and for non-medical imaging applications, such as in baggage security scanning and industrial inspection applications.

While the disclosure has been described in terms of specific embodiments, it is evident in view of the foregoing description that numerous alternatives, modifications and variations will be apparent to those skilled in the art. Each of the embodiments described herein can be implemented individually or in combination with any other embodiment unless expressly stated otherwise or clearly incompatible. Accordingly, the disclosure is intended to encompass all such alternatives, modifications and variations which fall within the scope and spirit of the disclosure and the following claims.

1. A detector structure, comprising:
a pixelated radiation sensor comprising a plurality of macropixels, each macropixel comprised of a plurality of sub-pixels; and
an anti-scatter grid (ASG) located over the pixelated radiation sensor, wherein a pitch between adjacent septa of the ASG along a first horizontal direction is less than 800 μm, and the septa of the ASG shield an equal number of peripheral edges of each sub-pixel of each of the macropixels of the pixelated radiation sensor.

2. The detector structure of claim 1, wherein a pitch between adjacent sub-pixels within each of the macropixels is between 300 μm and 400 μm.

3. The detector structure of claim 2, wherein the pitch between adjacent septa of the ASG along the first horizontal direction is 660 μm±20%, and the pitch between adjacent sub-pixels within each of the macropixels is 330 μm±20%.

4. The detector structure of claim 1, wherein each macropixel has a width of two sub-pixels along the first horizontal direction and a width of at least two sub-pixels along the second horizontal direction.

5. The detector structure of claim 4, wherein the ASG comprises a one-dimensional ASG comprising a plurality of septa extending parallel to one another along the second horizontal direction and laterally spaced from one another along the first horizontal direction, and each sub-pixel is partially shielded by a septum of the ASG along one peripheral edge of the sub-pixel.

6. The detector structure of claim 4, wherein the ASG comprises a two-dimensional ASG comprising a first plurality of septa extending parallel to one another along the first horizontal direction and a second plurality of septa extending parallel to one another along the second horizontal direction, and each sub-pixel is partially shielded by a septum of the ASG along two peripheral edges of the sub-pixel.

7. The detector structure of claim 1, wherein the pixelated radiation sensor comprises:
a direct conversion sensor material;
a cathode electrode over a first side of the direct conversion sensor material; and
a plurality of anode electrodes over a second side of the direct conversion sensor material, wherein each anode electrode defines a different sub-pixel, and the detector structure further comprises an application-specific integrated circuit (ASIC) electrically coupled to the anode electrodes of the pixelated radiation sensor and configured to generate photon count data for multiple energy bins for each of the sub-pixels.

8. The detector structure of claim 7, wherein the direct conversion sensor material comprises one or more of cadmium telluride, cadmium zinc telluride, cadmium selenide telluride, cadmium zinc selenide telluride, silicon, germanium, germanium arsenide, or a perovskite material.

9. The detector structure of claim 1, wherein a width dimension of each septum of the ASG is 80 μm or less.

10. The detector structure of claim 1, wherein the ASG comprises a plurality of first septa having a first vertical height dimension and a plurality of second septa having a second vertical height dimension, wherein the first vertical height dimension is greater than the second vertical height dimension.

11. The detector structure of claim 10, wherein:
the first septa and the second septa alternate with one another along the first horizontal direction;
the first vertical height dimension is greater than 2 mm and the second vertical height dimension is 1 mm or less; and
the second septa comprise at least one of tungsten, lead or molybdenum.

12. The detector structure of claim 10, wherein lower surfaces of the first septa are coplanar, and the second septa extend below a plane containing the lower surfaces of the first septa.

13. The detector structure of claim 12, wherein the ASG is a two-dimensional ASG, the second septa are located entirely below the plane containing the lower surfaces of the first septa and form a two-dimensional grid structure that underlies a two-dimensional grid structure of the first septa.

14. The detector structure of claim 10, wherein the second septa contact a surface of the radiation sensor.

15. The detector structure of claim 1, wherein at least one septum of the ASG comprises non-vertical sidewalls.

16. The detector structure of claim 15, wherein the at least one septum of the ASG comprises a tapered shape that is wider at an upper surface of the septum than at a bottom surface of the septum, and the non-vertical sidewalls extend between the upper surface and the bottom surface.

17. The detector structure of claim 15, wherein the non-vertical sidewalls comprise curved sidewalls such that the at least one septum has an S-shape in a side cross-section view.

18. An X-ray imaging system, comprising:
a radiation source configured to emit an X-ray beam; and
a detector array comprising a detector structure according to claim 1 that is configured to receive the X-ray beam from the radiation source through an intervening space configured to contain an object therein,
wherein the X-ray imaging system comprises a photon-counting computerized tomography (PCCT) imaging system comprising an image reconstruction system including a computer configured to run an automated image reconstruction algorithm on event detection signals generated by the pixel detectors of the detector array.

19. A detector structure, comprising:
a pixelated radiation sensor comprising a plurality of macropixels, each macropixel comprised of a plurality of sub-pixels; and
an anti-scatter grid (ASG) located over the pixelated radiation sensor,
wherein:
the ASG comprises a plurality of first septa having a first vertical height dimension and a plurality of second septa having a second vertical height dimension;
the first vertical height dimension is greater than the second vertical height dimension; and
the second septa contact a surface of the radiation sensor or the second septa protrude into a lateral space between the first septa.

20. The detector structure of claim 19, wherein the second septa contact the surface of the radiation sensor which comprises a surface of a cathode electrode of the radiation sensor.

21. The detector structure of claim 19, wherein the second septa protrude into the lateral space between the first septa such that a horizontal which extends from a sidewall of one first septa to a sidewall of another first septa cuts through a second septa located in the lateral space between the two first septa.

22. The detector structure of claim 19, wherein the second septa contact the surface of the radiation sensor, and the second septa protrude into the lateral space between the first septa.

23. A method of fabricating a detector structure, comprising:
forming an X-ray attenuating material over a surface of a pixelated radiation sensor; and
patterning the X-ray attenuating material located over the surface of the pixelated radiation detector to form a plurality of septa contacting the pixelated radiation sensor, wherein each septum extends between and partially shields pixel detectors of the pixelated radiation sensor.

24. The method of claim 23, further comprising providing additional septa over and vertically spaced from the surface of the pixelated radiation sensor, wherein the septa contacting the pixelated radiation sensor and the additional septa form an anti-scatter grid (ASG).

25. The method of claim 23, wherein the X-ray attenuating material comprises at least one of tungsten, lead or molybdenum.
